(19) United States
(12) Patent Application Publication
(10) Pub. No.: US 2018/0071139 A1
(43) Pub. Date: Mar. 15, 2018

(54) Body temperature regulating device

(71) Applicant: Clare P. Kobierowski, Cheltenham, PA (US)

(72) Inventor: Clare P. Kobierowski, Cheltenham, PA (US)

(21) Appl. No.: 15/330,361

(22) Filed: Sep. 12, 2016

(51) Int. Cl. A61F 7/02

(52) U.S. Cl. CPC A61F 7/02; A61F 2007/0035

(57) ABSTRACT

A body temperature regulating devise by use of a wrist band. The devise can be extended by adding the same devise and connecting them to each other to increase the length for neck, knee or leg. This devise uses small ice packs or frozen gel packs inserted into slotted pockets that will keep a person's body temperature lower than not using the devise. This body regulating devise has stretching elastic and adjustable “Velcro” straps to insure a satisfying fit.

### TECHNICAL FIELD

The present disclosure generally relates to a body temperature regulating device; and more particularly to a body temperature regulating device in the form of a band that conforms to a limb of the body.

### BRIEF DESCRIPTION OF THE DRAWINGS

FIG. 1 illustrates a perspective view of a body temperature regulating device, in a closed configuration, in accordance with an embodiment of the present disclosure;

FIG. 2 illustrates a planar side view of the body temperature regulating device of FIG. 1, in the closed configuration;

FIG. 3 illustrates a planar front view of the body temperature regulating device of FIG. 1, in an open configuration;

FIG. 4 illustrates a planar rear view of the body temperature regulating device of FIG. 1, in the open configuration;

FIG. 5 illustrates a planar view of the body temperature regulating device of FIG. 1 along its length, in the open configuration;

FIG. 6 illustrates a planar view of the body temperature regulating device of FIG. 1 along its length from opposite direction of FIG. 5, in the open configuration;

FIG. 7 illustrates a planar side view of the body temperature regulating device of FIG. 1, in the open configuration;

FIG. 8 illustrates a planar side view of the body temperature regulating device of FIG. 1 from opposite direction of FIG. 7, in the open configuration;

### DETAILED DESCRIPTION

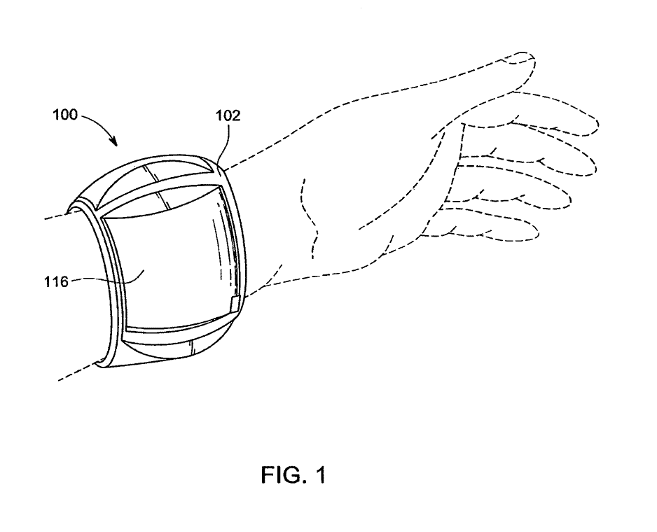

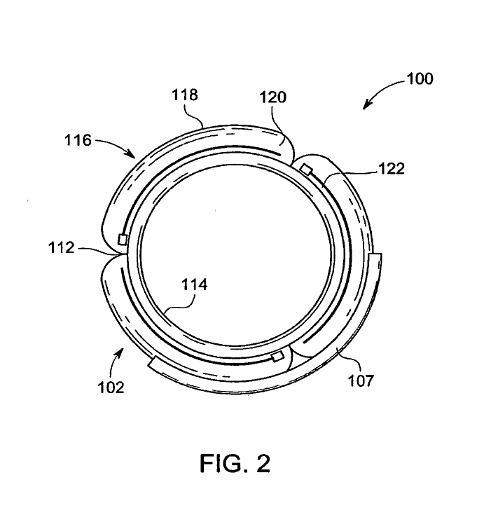

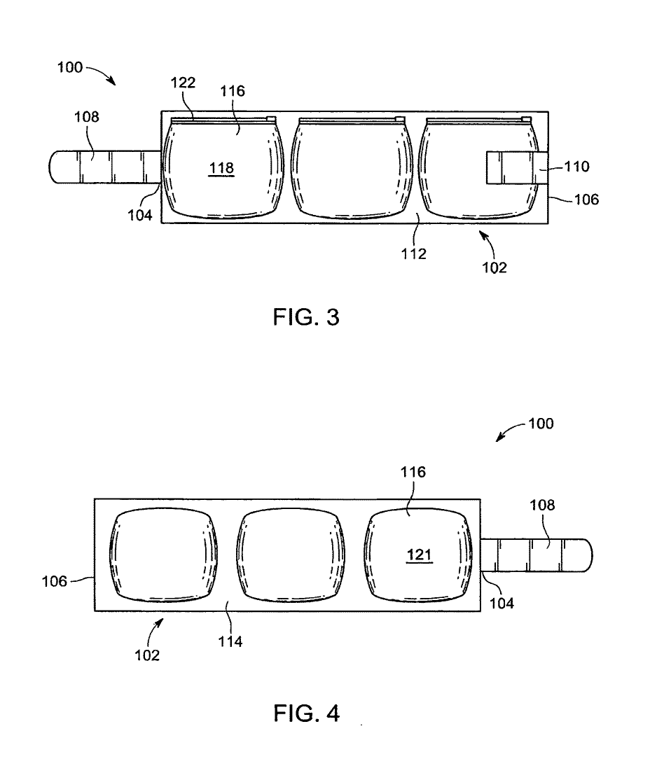

The present disclosure provides a body temperature regulating device (referenced by the numeral 100 and hereinafter sometimes simply referred to as device 100), as illustrated in the embodiments of FIGS. 1-8. Referring particularly to FIGS. 1-2, the body temperature regulating device 100 is shown in a closed configuration. Further, referring to FIGS. 3-4, the body temperature regulating device 100 is shown in an open configuration. The body temperature regulating device 100 includes a band 102 which is in the form of an elongated strip and, generally, constructed of a clothing material. For example, band 102 may be a rectangular shaped strip made of a breathable clothing material with sweat absorbing properties. The clothing material may be terrycloth, cotton, polyester, or any possible blend of the said clothing materials. Although it may be contemplated that any other suitable clothing material known in the art may be used with any limitations.

The band 102 may be constructed in different sizes to fit different wrist sizes or different body parts of a user. Band 102 is constructed in a manner to be lightweight and efficient in nature. It may be understood that band 102 is designed to flex in order to conform to the shape of the wrist of the user, or any other limb or body part, to be worn. Further, it may be understood that the band 102 is designed to stretch to a limited amount. In some examples, band 102 may be constructed of any stretchable material such as nylon, spandex, stretchable plastic, etc. Further, in one example, some sections of band 102 may be constructed of elastic or stretchable material. In accordance with the preferred embodiment of the disclosure, the elastic section includes a plurality of nylon strands to prevent stretching in a direction coaxial with the body part.

As better illustrated in FIGS. 3-4, the body temperature regulating device 100 is open at both its ends 104, 106. The device 100 includes a closing mechanism 107 (as illustrated in FIG. 2) for securing the two ends 104, 106 together, for example, in order to wrap device 100 over the wrist of the user. In an embodiment of the present disclosure, the closing mechanism 107 may include hook and loop fastener based means. Hook and loop fasteners are well known in the art and sold commercially under such product names as Velcro®. In such case, the closing mechanism 107 includes two securing pads 108, 110 located, respectively, at each of the two ends 104, 106. Further, the securing pads 108, 110 are placed in a manner such that a mating hook face of the securing pad 108 is disposed towards a front surface 112 of the band 102, and a mating loop face of the other securing pad 110 is disposed towards a back surface 114 of band 102; such that when the two ends 104, 106 are brought together, the surface pads 108, 110 mesh together to lock with each other and disposing the device 100 in the closed configuration.

As illustrated in FIGS. 3-4, the securing pads 108, 110 are shown as single pieces of hook and loop, essentially rectangular in shape; although it may be understood that the number and shape of such pads can vary. The closing mechanism 107 allows making band 102 adjustable and removable engaging with the wrist of the user. It may be contemplated that the closing mechanism 107 may, alternately, include a series of pins and holes, clasp and buckle, a latch, a zipper, snaps, or the like, that could be used in place of the securing pads 108, 110 for hook and loop fastener system for locking device 100 in the closed configuration.

In an embodiment of the present disclosure, the body temperature regulating device 100 includes a plurality of pockets 116 formed along a length of the band 102. In one example, each of the pockets 116 is substantially rectangular in shape with the longer side being parallel to the length of band 102. Further, a width of the pocket 116 is approximately equal to or slightly smaller than the width of band 102. It may be understood that in other examples, the pocket 116 may have any other suitable shape and/or size, such as, but not limited to, circular, square, triangular or any other polygonal shape. In the illustrated embodiment of FIGS. 1-8, device 100 is shown to include three pockets 116 arranged in series along the length of band 102. However it may be understood that in other examples, the device 100 may include more or less number of pockets 116 without any limitations. Further, as more clearly illustrated in FIG. 2, the gap between the pockets 116 is kept to a minimum in order to effectively cover the entire front surface 112 of band 102 for more efficient thermal cooling.

It may be understood that pocket 116 is a hollow structure and is designed to be pre-filled/accommodate a source of thermal energy (not shown), hereinafter referred to as “thermal pack” (not shown). Basically, the thermal pack is any fluid or gel with high heat storing capacity. The in one embodiment, the thermal packs used in different embodiments of the present disclosure are such as, but not limited to, Propylene Glycol packs (or any similar material). Such thermal packs are manufactured by electro-sonic welding techniques for fusing plastic sheets together on three sides, and then filling the pack with the Glycol liquid and sealing the fourth side. Electronic welding of plastic seams is preferred over conventional “heat bar” welding because it provides a stronger, more leak-proof weld. Ultrasonic plastic welding is achieved by vibrating a metal horn at high frequency such that heat is generated throughout the material, which in turn gets welded by sheer friction. Since the vibrations saturate the plastic being welded, all of the plastic becomes equally and evenly hot providing a very uniform solid weld.

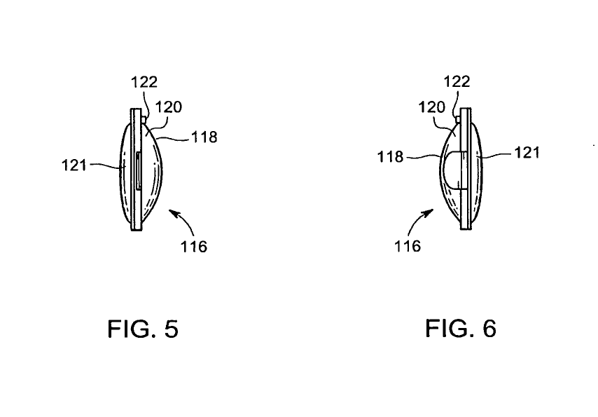

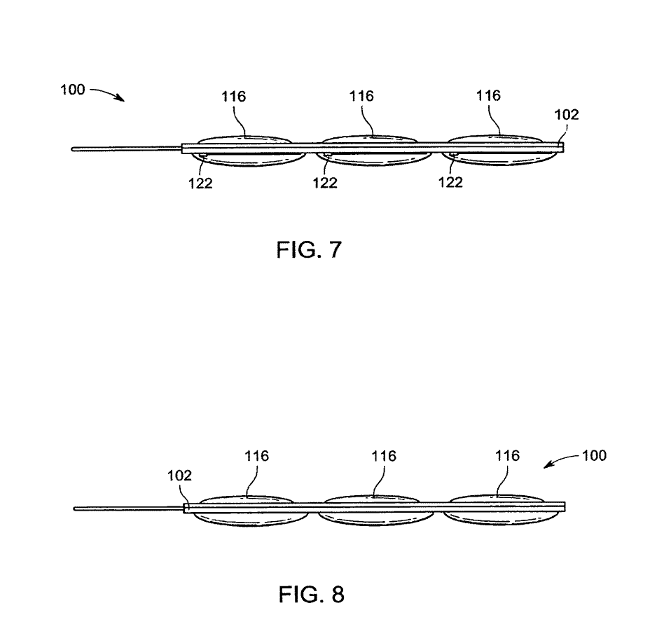

As more clearly illustrated in FIGS. 5-6 and further in FIGS. 7-8, the shape of the pocket 116 is substantially oblong extending orthogonally above the front surface 112 of band 102, defining a top face 118 and two side faces 120. In some examples, pocket 116 may further extend from the back surface 114 of band 102 to impart a substantially spherical shape to pocket 116 and defining a bottom face 121 (more clearly shown in FIG. 4 and referenced by numeral 121), 4). In other examples, pocket 116 may have a substantially flat bottom face 121 to generally conform to the shape of the body part wrist or other body part of the user to which the device 100 is worn. The ability of pocket 116 to conform to the shape of the body part also reduces the possibility device 100 from slipping on the body part. It may be contemplated that the overall volume of hollow pocket 116 is greater than the volume of the thermal pack to easily accommodate the thermal pack. Further, volume of the hollow pocket 116 is designed to be sufficient enough to compensate for any increase in volume of the thermal pack due to heating thereof.

In some examples, pocket 116 is made of same material as band 102. Another variation of general embodiment contemplates an insulating means between the thermal pack, and faces 118, 120 of pocket, and 116 which do not directly contact the body. That is, top face 118 and the side faces 120 of pocket 116 may be made of insulating material so that top face 118 and the side faces 120 may have a lower thermal conductivity, in order to reduce and thus minimizes the thermal loss from the thermal pack inside pocket 116 to external environment, whereas bottom face 121 may be made of breathable and conductive material, generally same as that of band 102. One insulating material contemplated is a type of semi-rigid foam plastic made from polystyrene, polyurethane or any other material known in the art.

In one embodiment of present disclosure, thermal packs are removably disposable in pockets 116, that is, thermal packs can be removed from pocket 116 for cleaning and/or for re-cooling. For this purpose, each pocket 116 is provided with a plastic zipper 122 (also known as “Ziploc™”) disposed along one of the side faces 120 and running parallel along the longer side of band 102. Such plastic zippers 122 are widely known in art and have not been described in detail herein for the brevity of present disclosure. It may be understood that the plastic zipper 122 allows for substantially and hermetic sealing of pocket 116 and thus prevent any leakage of fluid from thermal pack to outside of pocket 116. It may be contemplated that, in alternate examples, some other sealing means may be employed for sealing pocket 116 without departing from the scope of the present disclosure.

An alternative embodiment contemplates that pockets 116 may be permanently sealed after providing thermal packs inside pockets 116, and thus does not permit removal of thermal packs by the user. In this embodiment, the open end of pocket 116 is sealed once the thermal pack is inserted, thus eliminating the need for the plastic zipper 122 as discussed above. In such case, the thermal pack would be regenerated by heating or chilling the entire device 100 instead of the thermal pack itself. For example, pockets 116 can be filled with water and frozen to activate the cooling properties of device 100. Due to the permanent seal, pocket 116 holds the fluid in the interior thereof during the freezing and thawing of fluid. In yet other examples, pocket 116 may be made of water-proof materials such that the user can easily fill pocket 116 of the device 100 by removing the thermal pack from the pocket 116 and filling it with loose fluid, such as water from a pitcher, or faucet. The user can then place device 100, with pocket 116 full of fluid, in the freezer, and the fluid will freeze in a short amount of time, thereby freezing device 100.

In one example, two securing pads 108, 110 may be sewn to band 102 at its ends of 104, 106. In other example, as illustrated in FIG. 3, the plurality of pockets 116 substantially covers the front surface 112 of band 102, and at least some portion securing pads 108, 110 may be located over t pocket 116. Closing mechanism 107 is used and needed to hold the parting sides together once the users wraps device 100 around their wrist, whereby securing pads 108, 110 attaching in an end-to-end manner. It may be understood that multiple devices 100 may being combined by locking their corresponding securing pads together, and thus increasing the overall length of device 100. This may be helpful when the user needs to use device 100 for a larger part of the body; for example, from using the single device 100 on a wrist to combining multiple devices to be used around larger parts of the human body such as the neck, waist, and ankle or howsoever the user would like to cool down a particular part of the body.

Device 100 is placed over the pulse of wrist by the user placing their hand through band 102. The device 100, with pockets 116 filled with the thermal packs, can be placed on the underside of the user's wrist at the base of the palm. In this position device 100 has significant cooling potential, due to the close proximity to the pulses. Device 100 of the present disclosure can be used by athletes, outdoor workers, and any user who is exposed to extreme heat. Device 100 helps a user keep cool and avoid heat exhaustion during exercise or strenuous work. This may allow a user to work in a hot or humid environment for longer time periods. A user can also take device 100 in a thermally insulating enclosure to keep device 100 cool as needed. Further, device 100 works like a regular wristband by absorbing sweat with its terrycloth structure. It may also be contemplated that the device 100 could alternatively be used for heating purposes without departing from the spirit of the present disclosure.

While certain exemplary embodiments have been described and shown in the accompanying drawings, it is to be understood that such embodiments are merely illustrative of and not restrictive on the broad present disclosure, and that this present disclosure is not limited to the specific constructions and arrangements shown and described, since various other modifications and/or adaptations may occur to those of ordinary skill in the art. It is to be understood that individual features shown or described for one embodiment may be combined with individual features shown or described for another embodiment.

### Body Temperature Regulating Device

### References Cited—U. S. Patent Documents

| | | |
|---|---|---|
| 9,301,557 B1 | April 2016 | Elmer Santos |
| 2013/0025315 A1 | January 2013 | Robert Anthony Freeman |
| D684,497 S | January 2013 | Nike, Inc. |
| 8,474,284 B2 | July 2013 | Ryan Rush |
| 8,327,566 B2 | December 2012 | Bryce C. Waggoner |
| 8,276,298 B2 | October 2012 | John M. Padgett |
| 8,250,796 B2 | August 2012 | John M. Padgett |
| 8,225,626 B1 | July 2012 | Eddie Blau |

1. A wrist band with zipper locked pockets to hold cold or hot packs inside.

2. A wrist band made to attach to another to extend a required length.
